(19) United States
(12) Patent Application Publication
(10) Pub. No.: US 2007/0066872 A1
(43) Pub. Date: Mar. 22, 2007

(54) IIIuminable retractor

(76) Inventors: Paul Morrison, Christchurch (NZ); Gray Felix Heynen, Auckland (NZ)

Correspondence Address: JACOBSON HOLMAN PLLC, 400 SEVENTH STREET N.W., SUITE 600, WASHINGTON, DC, 20004, US

(21) Appl. No.: 10/553,805

(22) PCT Filed: Mar. 8, 2004
(86) PCT No.: PCT/IB04/50207

§ 371 Date: Nov. 27, 2006

(30) Foreign Application Priority Data
Mar. 7, 2003 (NZ) 524638
Aug. 12, 2003 (NZ) 527576

(51) Int. Cl. A61B 1/32

(52) U.S. Cl. 600/212; 600/245

(57) ABSTRACT

A retractor assembly suitable for hip or other joint surgery comprising a structural member (1), which consists of a handle (2) that extends proximal through a curved region (4) to a distal region (3) adapted for use in a suitable patient, a light ducting member (5), which is at least in part in substantial conformity to the curved region (4), adapted in use to receive light and reliant on internal reflection of the light, to transmit such received light about at least part of the curved region to an emission outlet (7) which casts light to a zone at and/or about said distal region (3), and a shield (6) overlying at least that part of the light ducting means (5) in substantial conformity to at least part of the curved region (4), wherein the light ducting member (5), the shield (6) and the structural member (1) can be brought into assembly so that directly and/or indirectly each of the light ducting member (5) and the shield (6) is in a supported condition relative to the structural member (1).

This is a nationalization of PCT/IB04/050207 filed Mar. 8, 2004 and published in English.

### TECHNICAL FIELD

The present invention relates to a retractor useful for minimally invasive and minimal incision hip surgery.

### BACKGROUND ART

With surgery of the kind referred to above (whether for hip replacement or other purposes) there is a need for a retractor. Unfortunately with such a retractor and its size relativity to the incision there is a negation of the effectiveness of any overhead lighting.

Therefore for this purpose we believe a source of light (preferably of very low profile) associated with the retractor will better allow vision of the distal end regions of the retractor.

Various retractors have been proposed. See for example, U.S. Pat. Nos. 5,928,139 and 6,139,493 and PCT Publication WO 02/19919.

The present invention however recognises a need for a retractor (suitable for such hip surgery) [whether an anterior retractor or otherwise] preferably of low profile yet having an illuminable distal retraction end linked to a handle capable of being manipulated during surgery.

### DISCLOSURE OF THE INVENTION

In a first aspect the invention consists in a retractor assembly suitable for hip or other joint surgery, the assembly comprising or including

a structural member capable of being used as a retractor, the structural member having, as a proximal region, a handle or manipulation control region (“handle”) extending through a curved region to a distal region adapted for use in a suitable patient,

a light ducting member or assembly of members (“light ducting means”) at least in part in substantial conformity to the curved region adapted (with respect to the structural member with a light inlet nearer the proximal region than the distal region) in use to receive (preferably ducted) light and reliant on internal reflection of the light, to transmit such received light about at least part of the curved region to an emission outlet or outlets (“light outlet”) which will cast light to a zone at and/or about said distal region, and

a shield overlying at least that part of the light ducting means in substantial conformity to at least part of the curved region,

wherein the light ducting member, the shield and the structural member can be brought into assembly so that directly and/or indirectly each of the light ducting member and the shield is in a supported condition relative to the structural member.

As used herein “curved” in respect of the “curved region” can mean any appropriate angular transition from one end to the other end thereof whether regular or irregular and whether continuous or intermittent or otherwise.

As used herein “supported condition” means supported against accidental easy skewing or separation.

As used herein the term “and/or” has the meaning “and” or “or” and, where the context might allow, both.

As used herein “(s)” at the end of a word means either or both the singular and plural forms of the word.

Preferably the supported condition provides directly and/or indirectly a support of both a proximal and distal region of the ducting member and/or both a proximal and distal region of the shield.

In some forms of the present invention indirect support for the light ducting member might be provided by a direct support by the structural member of the shield which by overlying the light ducting member thus provides support therefore relative to the structural member.

In some forms such direct and/or indirect support can involve the light ducting means, whether as a member or an assembly of members, being engageable to the structural member by any appropriate means at or adjacent a distal region of the light ducting means. Examples include a clip-fit arrangement, a slide on arrangement or any alternative.

Options of such direct or indirect engagement of a light ducting member to the structural member, the shield to the structural member and the light ducting member to the shield are disclosed hereinafter and all such forms of relationship and any combination of them falls within the scope of the present invention.

Preferably the light ducting means at least from its light inlet to its light outlet is a moulded member (of one or more material(s)) having a surface adapted to internally reflect light so as to favour ducting of light from the inlet to the outlet.

Preferably the outlet is an outlet that issues as a beam or beams a band of light rather than a more consolidated area of light, e.g.; preferably the light inlet is adapted to receive light from a fibre optic bundle, a light cable or the like and may be, for example, of a circular area whilst the surfaces surrounding the light path from the inlet to the light outlet is such as to duct the light to the more banded form light outlet. Alternatively the assembly can include a light source such as a LED or LEDs.

Preferably any such banded outlet conforms more to the transverse extent of the structural member at, at least the more distal part of the curved region.

Preferably the curved region has a major transverse axis of the structural member normal to the curving thereby to provide a low profile for the curve region.

As used herein the term “low profile” refers to a retractor or retractor assembly or components thereof better able to provide less of a profile transversely down through the curved region than it provides in other directions, i.e.; substantially as hereinafter described by reference to preferred embodiments.

Preferably the light ducting means is an assembly of a moulded plastics component having the light inlet and the light outlet(s) and a means of association thereof (e.g.; attachment apparatus as hereinafter referred to) to the structural member.

In another aspect the invention consists in a retractor assembly suitable for hip or other joint surgery, the assembly comprising or including

a structural member capable of being used as a retractor, the structural member having, as a proximal region, a handle or manipulation control region (“handle”) extending through a curved region to a distal region adapted for use in a suitable patient,

a light ducting member or assembly of members (“light ducting means”) at least in part in substantial conformity to the curved region adapted (with respect to the structural member with a light inlet nearer the proximal region than the distal region) in use to receive (preferably ducted) light and reliant on internal reflection of the light, to transmit such received light about at least part of the curved region to an emission zone (“light outlet”) which will cast light to a zone at and/or about said distal region, and

a shield overlying at least that part of the light ducting means in substantial conformity to at least part of the curved region,

wherein the light ducting member, the shield and the structural member can be brought into assembly so that directly and/or indirectly each of the light ducting member and the shield is in a supported condition relative to the structural member.

In a further aspect the invention consists in a structural member of a retractor assembly as aforesaid.

In a further aspect the present invention consists in light ducting means (as defined previously) as aforesaid.

In yet a further aspect the present invention consists in a shield suitable for a retractor assembly as aforesaid.

In a further aspect the invention is a sterile light ducting means as aforesaid in a pack therefor.

In a further aspect the present invention consists in, in combination, as a sterile pack, light ducting means and a shield as aforesaid.

In a further aspect the present invention consists in a method of providing a retractor assembly as aforesaid, said method comprising or including taking a light ducting means (and preferably also a shield) from a pack and associating the same with the structural member and the shield.

Preferably the structural member is sterile prior to such association of the light ducting means and shield therewith.

Preferably the light ducting means and optionally also the shield is disposable.

In another aspect the present invention consists in a retractor useful in surgery (e.g.; hip surgery), said retractor having

a main structural member defining

a handle region,

a distal region, and

an intermediate region, said intermediate region curving on planes normal to its main transverse dimension thereby to define a low profile form having a “concave” lower side and a “convex” upper side,

a light duct capable of ducting light it receives, said duct, at least in part away from its handle proximate inlet end, substantially conforming to the intermediate region so as to maintain a low profile thereover whilst having an emission end capable of emitting light the duct has received towards a zone in which said distal region of the main structural member is being operated,

attachment apparatus integral with or attaching to the light duct at or adjacent the inlet end of the light duct engaged with or for engagement with the main structural member, and

a shielding member attachable to at least one of
i) the attachment member,
ii) the light duct, and
iii) the intermediate member

so as, also in a low profile at least in part away from its handle proximate end, of substantially conforming to the light duct and/or the adjacent intermediate region.

As used herein the terms “convex” and “concave” are not used in any strict geometrical sense but rather as a means of conveying a continuous or discontinuous curving or some variation thereof where there is some degree of surface to surface concordance better to provide a low profile whilst ensuring the angular offset needed for retraction use.

Preferably the light duct is a moulded transparent plastics member preferably having the attachment integrally moulded therewith. Alternatively preferably the light duct is a fabrication from two moulded components.

Preferably said light duct is adapted at its inlet end to receive light ducted via light cable, a fibre optic bundle, tube, light cable or the like.

Preferably the ratio between the light inlet surface area and light outlet surface area is a ratio of no less than 1:1 and no more than 1:11 The current preferred designed ratio is 1:2.2

Preferably the emission end of the light duct is substantially of a flattened section so as to better conform to the low profile of the intermediate region.

Preferably the ratio between the light inlet surface area and light outlet surface area is a ratio of no less than 1:1 and no more than 1:11 The current preferred designed ratio is 1:2.2

Preferably the flattening and broadening is such that the outlet is thinner than the inlet diameter or notional diameter and is at least as wide as two such diameter (preferably is three or more such diameters in width).

Preferably the light duct splays to said flattened form from a non flattened form at the inlet end.

Preferably said shielding member has a form adapted to conform closely to the flattened form of the light duct.

Preferably said main structural member, light duct, attachment apparatus and/or shielding member is substantially as hereinafter described with reference to any one or more of the accompanying drawings.

In another aspect the invention consists in a retractor assembly suitable for hip or other joint surgery, the assembly comprising or including

a structural member capable of being used as a retractor, the structural member having, as a proximal region, a handle or manipulation control region (“handle”) extending through a curved region to a distal region adapted for use in a suitable patient, and

a light ducting member or assembly of members (“light ducting means”) at least in part in substantial conformity to the curved region adapted (with respect to the structural member with a light inlet nearer the proximal region than the distal region) in use to receive (preferably ducted) light and,g reliant on internal reflection of the light, to transmit such received light about at least part of the curved region to an emission outlet or outlets (“light outlet”) which will cast light to a zone at and/or about said distal region, and

wherein the light ducting member and the structural member can be brought into assembly so that the light ducting member and the shield is in a supported condition relative to the structural member.

In still a further aspect the present invention consists in a retractor in accordance with the present invention useful in hip surgery, said retractor capable of being assembled and disassembled by attachment and removal respectively of components. In some forms some of the components can be disposable but preferably each is capable of being cleaned for reuse.

In still a further aspect the present invention consists in the use of a retractor assembly or retractor in accordance with the present invention, the inlet end of the light transmitter receiving a feed of light to illuminate a zone in which the distal region of the main structural member is being operated.

In still a further aspect the present invention consists in a method of treating a mammalian patient (whether a human being or otherwise) which involves the operative use of a retractor assembly or retractor of the present invention as a retractor.

Preferably said method involves feeding light into the light transmitter or duct thereby to illuminate part of the patient (e.g.; during a hip replacement operation).

In another aspect the present invention consists in a retractor useful in surgery (e.g.; hip surgery), said retractor having

a main structural member defining

a handle region,

a distal region, and

an intermediate region, said intermediate region curving on planes normal to its main transverse dimension thereby to define a low profile form having a “concave” lower side and a “convex” upper side,

a light duct or ducts capable of ducting light it receives, said duct(s), at least in part away from its (their) more handle proximate inlet end, substantially conforming to the intermediate region so as to maintain a low profile thereover whilst having an emission end capable of emitting light the duct has received towards a zone in which said distal region of the main structural member is being operated,

attachment apparatus integral with or attaching to the or each light duct at or adjacent the inlet end of the light duct(s) engaged with or for engagement with the main structural member,

a shielding member attachable to at least one of
i) the attachment member,
ii) the light duct(s), and
iii) the intermediate member

so as, also in a low profile at least in part away from its handle proximate end, of substantially conforming to the light duct and/or the adjacent intermediate region.

In another aspect the invention consists in a retractor assembly suitable for hip or other joint surgery, the assembly comprising or including

a structural member capable of being used as a retractor, the structural member having, as a proximal region, a handle or manipulation control region (“handle”) extending through a curved region to a distal region adapted for use in a suitable patient,

at least one light ducting member (“light ducting means”) carried directly or indirectly by the structural member with a light inlet in use to receive light and, reliant on internal reflection of the light, to transmit such received light to an emission zone (“light outlet”) which will cast light to a zone at and/or about said distal region, and

at least one LED or other light source carried directly or indirectly by the structural member and positioned to provide light inputs into the inlet(s) of the light ducting means, and

provision for mounting a battery and completing the operating circuit of the LED(s) or other light source(s) (e.g. a dock and cover (and optionally a switch)), (optionally) a shield overlying at least part of the light ducting means,

wherein when assembled the light ducting means can illuminate the and/or about the distal region.

In another aspect the invention consists in a retractor assembly suitable for hip or other joint surgery, the assembly comprising or including

a structural member capable of being used as a retractor, the structural member having, as a proximal region, a handle or manipulation control region (“handle”) extending through a curved region to a distal region adapted for use in a suitable patient,

a light ducting member or assembly of members (“light ducting means”) at least in part in substantial conformity to the curved region adapted (with respect to the structural member with a light inlet or light inlets) in use to receive light and, reliant on internal reflection of the light, to transmit such received light about at least part of the curved region to an emission outlet or outlets (“light outlet”) which will cast light to a zone at and/or about said distal region,

at least one LED or other light source carried directly or indirectly by the structural member and positioned to provide light inputs into the inlet(s) of the light ducting means,

provision for mounting a battery and completing the operating circuit of the LED(s) or other light source(s), and

optionally a shielding member for at least part of the light ducting means, and

optionally a cover for a battery forming at least part of said provision for mounting a battery and completing the operating circuit.

In still another aspect the invention is a surgical retractor having

a retractor body,

at least one light duct carried (directly or indirectly) by the retractor body,

a LED or other light source carried (directly or indirectly) by the retractor body for the or each light duct,

a battery powered circuit carried by the retractor body (directly or indirectly), and

a battery mounting for battery location in the circuit,

and optionally a shielding member for at least part of the light duct(s),

wherein the retractor has a handling end and a body insertion end and the light duct(s) is (are) adapted to beam light received from the LED(s) and/or other light source(s) to and/or about the body insertion end of the retractor body.

The invention is also the related uses, methods, components and subassemblies of such battery powered versions.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

A preferred form of the present invention will now be described with reference to the accompanying drawings in which:

FIG. 1 shows a partial sub-assembly of an anterior retractor suitable for use in hip replacement operations, the sub-assembly showing attached by a clip or attachment apparatus a light duct (preferably of solid transparent material) spreading to a low profile form (i.e.; from its handle proximate light inlet end to its beam directing outlet end) adapted to bathe a zone adjacent the distal region shown in a band of light or light beams, the shield being shown exploded off the assembly but showing how that too maintains a low profile over the regions of the assembly likely to be in or close to being in a patient during the course of an operation,

FIG. 2 shows various views of a light duct or pipe (but of solid material so as to “pipe” by internal reflection of the light),

FIG. 2A showing a side view of the apparatus and showing its curved region to conform and thus provide a low profile in conjunction with the structural member depicted in FIG. 1,

FIG. 2B is a view AA of the apparatus of FIG. 2A,

FIG. 2C is a section at BB of FIG. 2A,

FIG. 2D is a section at CC of FIG. 2A,

FIG. 2E is an enlargement of part of the detail of FIG. 2D to show the nature of the retention characteristic of the moulded outstand of the light duct,

FIG. 2F is a view from below of the member of FIG. 2A showing the offsetting of the inlet end for light from the wide but narrow beam producing outlet end,

FIG. 3A is a side elevation of the shield member shown in much the same orientation as is the light duct in FIG. 2A,

FIG. 3B is an end view of the shield of FIG. 3A in the direction CC,

FIG. 3C is a section of the shield at DD,

FIG. 3CC is a larger detailing of the section of FIG. 3C,

FIG. 3D is a sectional view at EE of the shield of FIG. 3A,

FIG. 3DD is a larger detailing of FIG. 3D,

FIG. 3E is a bottom view of the shield of FIG. 3A showing the corresponding offset to that of the light duct shown in FIG. 2E,

FIG. 4A is a pictorial view of attachment apparatus or a clip for (e.g.; push fit) association with the light duct and for engagement by a clip-fit arrangement to the handle or the transition of the handle into the curved region of the structural member,

FIG. 4B is a plan view of the attachment apparatus of FIG. 4A,

FIG. 4C is a view FF of the apparatus of FIG. 4B,

FIG. 4D is a section at GG of the clip or attachment apparatus of FIG. 4C,

FIG. 4E is a view HH of the apparatus of FIG. 4B,

FIG. 4F is a view II of the apparatus of FIG. 4B,

FIG. 4FF is an enlargement of part of the detail of FIG. 4F showing details of the clip fit retention shoulder forming one part of the clipping feature of the attachment apparatus,

FIG. 5 shows the relationship of the shield of FIG. 3A through 3DD with its associated light duct as shown in FIGS. 2A through 2E,

FIG. 6 is an end view of the arrangement of FIG. 5 showing how retention shoulders on either side of the shield are available for attaching the shield and thus assisting in supporting the outlet end of the light duct relative to the structural member (not shown),

FIG. 7, in a similar manner to that of FIG. 6, shows how, if desired, the protuberant portion of the light duct of FIGS. 2A through 2E can locate through an opening of the structural member,

FIG. 8 shows part of a structural member showing how, if desired, a plurality of different openings can be provided therein at the curved region or immediate region or approaching the distal end so as to allow engagement members such as those down standing from the shield as shown in FIG. 6 to engage without going outside of the shield edges,

FIG. 9 shows a view of a structural member having a variety of openings therein for location or engagement purposes,

FIGS. 10A and 10B show respectively an attachment and detachment of, for example, a clip fit arrangement internally of an opening at or adjacent the transition from the handle into the curved region or intermediate region, the attachment and releasement mechanisms being shown by the arrows,

FIG. 11 and FIG. 12 show options with respect to some of the openings through the structural member depicted in FIG. 9 for location of the shield, and

FIGS. 13 and 14 are respectively other views demonstrating the relationship that arises from the engagements of FIGS. 11 and 12 respectively.

FIG. 15 is a battery powerable variant of an illuminated surgical retractor for use in minimally invasive hip surgery, and

FIG. 16 shows in a part exploded version some components and, in some sub assembly, the rest of the assembly of FIG. 15.

### BEST MODE FOR CARRYING OUT THE INVENTION

Component List: (When Input Light is from a Light Cable):

Retractor;
This could be any retractor, size or shape not critical. Our design as detailed in the drawings is for mini incision and minimally invasive hip replacement.
Manufactured from any suitable material i.e. plastic, carbon fibre, stainless steel, titanium.

Light Duct;
Clip, this is the attachment point for the light cable and pipe to retractor.

Attachment to the cable is in the form of a screw connection or push lock type. Material could be plastic (e.g.; ABS, Acetyl, polycarbonate, etc.) or metallic. In some situations this feature could be permanently attached to the retractors. Where the shield is utilised to retain the light duct the role of the clip could be reduced to that of attachment interface between the light duct and light cable or may not be required when the cable detail is moulded directly to the pipe. The clip will in some variants provide some or all of the attachment to the retractor through tabs which locate in detail on the surface of the retractor or wrap around the retractor.
Duct, is moulded from a transparent material (i.e. plastic (i.e.; acrylic, polycarbonate, etc.) or glass) with a high gloss finish over the entire part. Some variants may have a shaped or textured light exit face to change the light pattern emitting from the duct. It may be provided with an exterior layer or coat that does not cover the inlet or outlet.
Shield, this forms a protective barrier to reduce the chance of damage to the duct from other instruments. It may also improve structural integrity.

In some variants this will be optional and may be supplied separately to be placed on the duct during or pre use. The shield attaches to the pipe through detail on the shield which form undercuts or with sprung clipping features. Material can be any durable plastic or a metallic material (ductile or high tensile) [e.g.; stainless steel, spring steel, etc.].

The preferred arrangement with the various engagement options discussed is such that a structural member 1 defines a handle region 2 which has a distal end region 3 linked thereto by a curved region 4 or intermediate region 4 which is of low profile, i.e.; wider than it is thick so that the reduced thickness transversely down through the curved region provides the low profile. Likewise for the light duct 5 and the shield 6 for regions to substantially conform to at least part of the curved region to the extent required to allow the outlet end 7 of the light duct 5 to direct its beam or beams, as a band, towards the distal end region 3 or the zone of a patients body in proximity thereto.

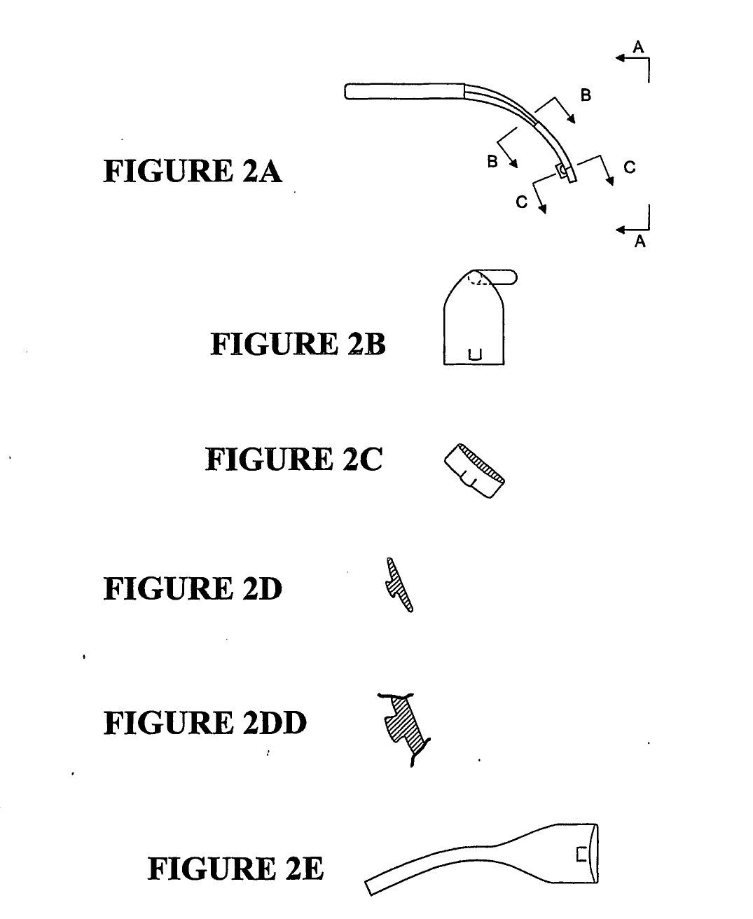

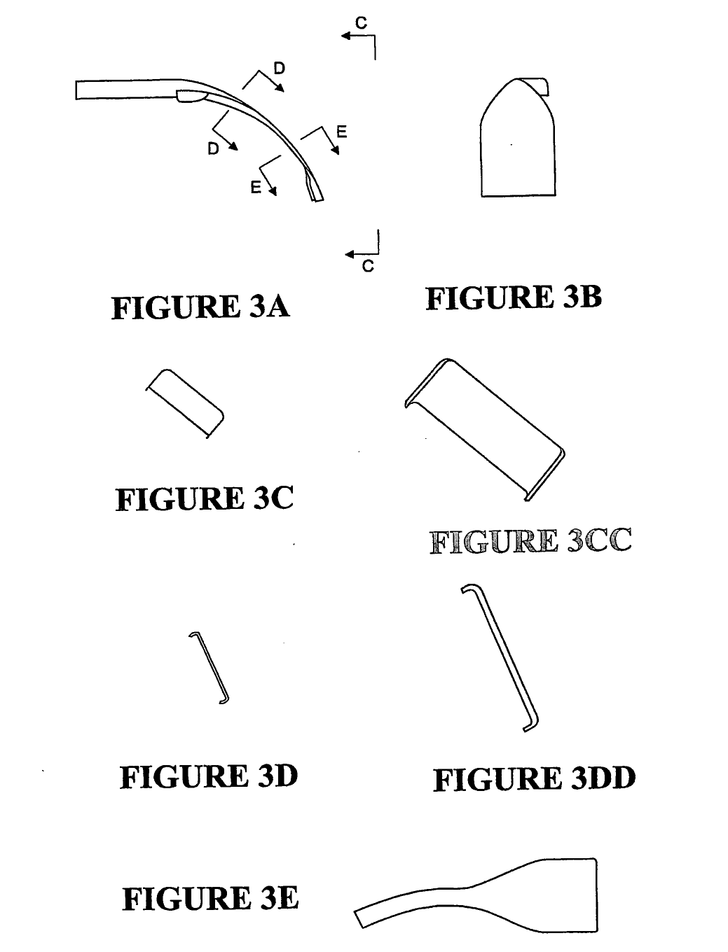

As will be apparent from the drawings the light member 5 can be provided as the moulded member shown in FIGS. 2A through 2E as a member that is distinct from engagement apparatus 8 adapted to engage by a thread or other means 9 to the end of a light cable so that the push fitted inlet end of the received light duct (preferably round in section and offset as shown) can take in a high lux beam and offer out as a band (at some reduction in lux) the illuminating beam.

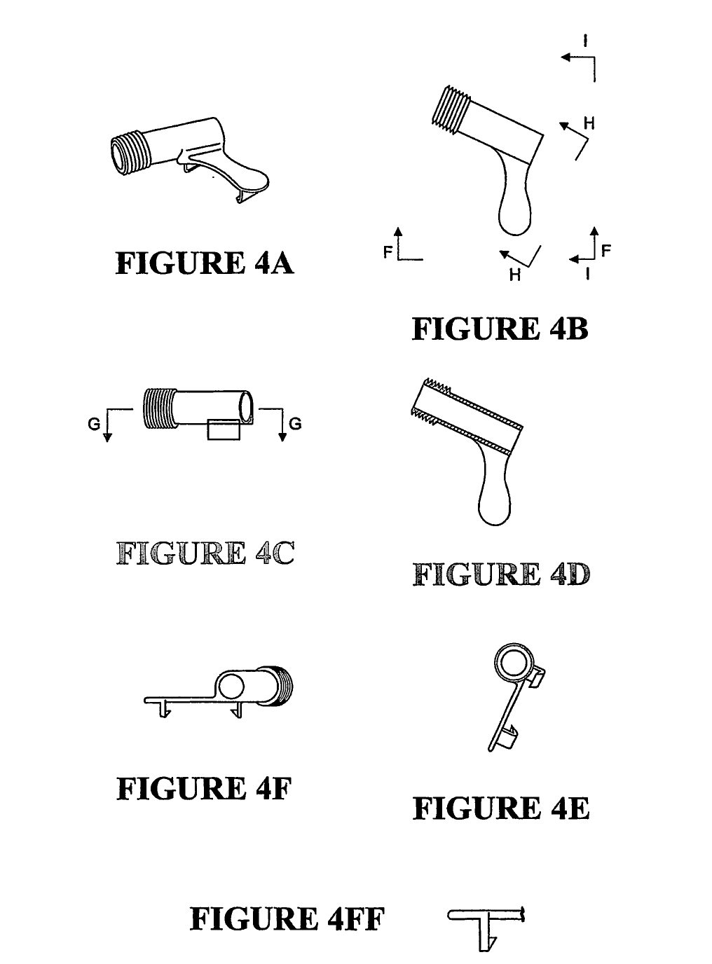

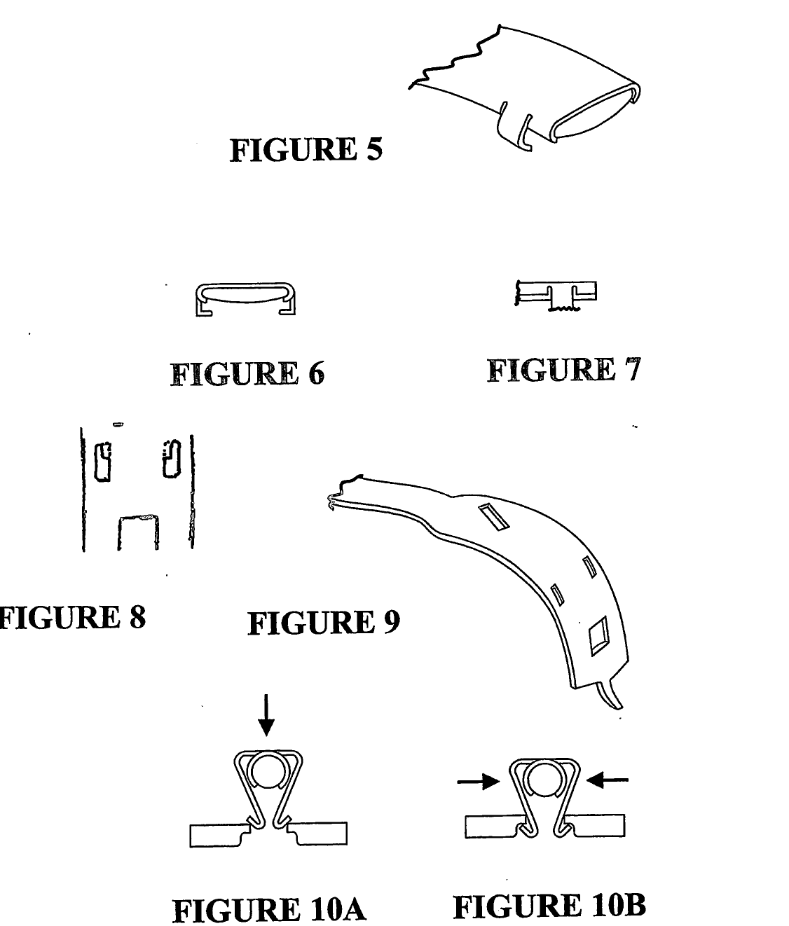

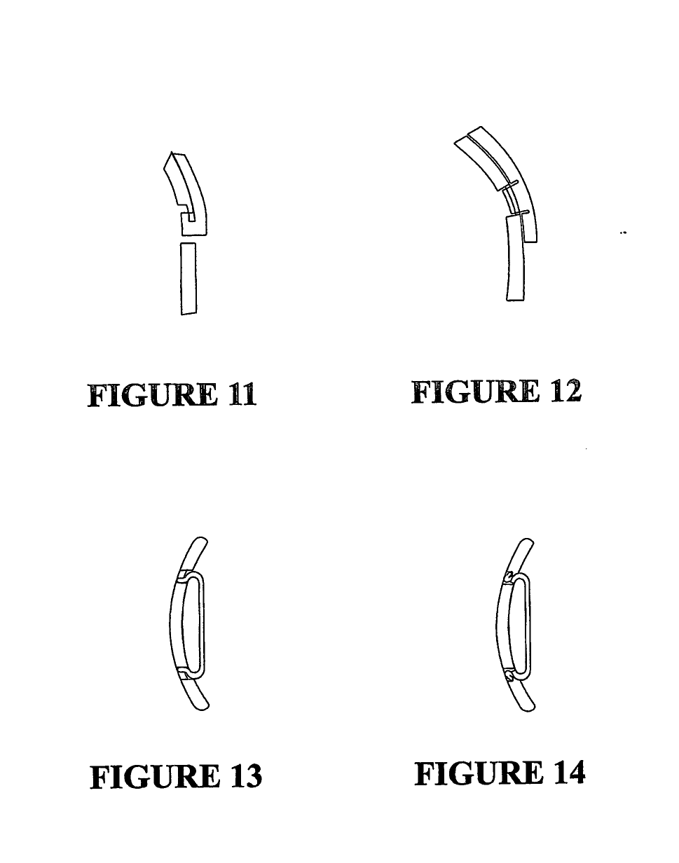

The clamp, clip or the like 8 can of course be integrally moulded with the light tube 5 and have such feature as is apparent from FIGS. 4A through 4FF as will allow clip fit engagement to the handle 2 of the structural member 1.

Any appropriate clip fit, sliding or permanent fitting of such attachment apparatus 8 can be contemplated and various options for engagement of components together to provide an assembly of a disposable light duct and means for attachment thereof to the structural member at or adjacent the transition of the handle into the curved region can be contemplated. Likewise any association directly or indirectly of other regions of the light duct such as by some measure of location as depicted in FIG. 7 as a result of the protuberance shown in FIG. 2DD and an appropriate opening therefore in the structural member as shown in FIG. 7.

The present invention is adapted to stabilise the light duct against skewing by appropriate direct or indirect, or both, interactions.

The present invention contemplates situations where the shield can be provided as a separate item (e.g.; supplied with a disposable light duct) or be kept with the structural member 2. Similarly for the member 8.

Disposable light members 5 can be utilised or they can be recycled for use if not damaged.

Any appropriate arrangement whereby there is the assembly, disassembly arrangement contemplated by the earlier statements of the invention are within the scope of the present invention as there are alternative arrangements reliant on such means of inter-engagement which may obviate the need for a shield as a separate item if the light duct itself does not provide concerns as to damage of an exposed surface and can be stabilised using any one or more of the interactions herein depicted of described.

The light duct can sit on the top surface of the structured member. It can be fixed to the structural member through moulded detail on the light duct and clip locking into detail on the structural member. The low profile and close fit provides good visual access.

The light duct alternatively can sit on the top surface of the structural member and be fixed through moulded detail on the light pipe and clip locking into detail on the structural member.

The low profile and close fit provides good visual access.

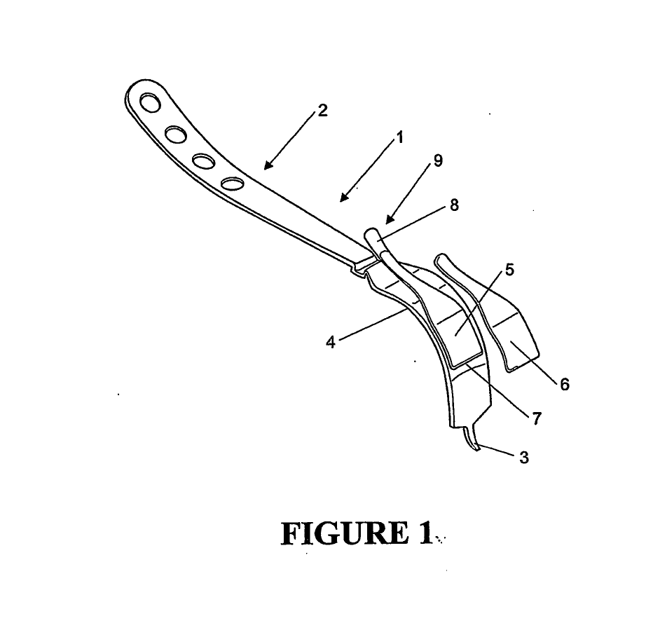

Reference has been made to the light duct preferably being on the convex side as shown in FIG. 1 of the curved region. Other possibilities include its location on the concave side or part of both and, if desired, provision can be made for outlet or outlets from protruding parts of the light duct for the light through openings of the curved region that nonetheless still display light to the same region as will the outlet 7 shown in FIG. 1. Multiple outlets for that purpose each from a variety of different poke through parts of a light duct can be contemplated. Such forms however may be less desirable owing to a greater loss of received ducted light.

Preferably the ratio between the light inlet surface area and light outlet surface area is a ratio of no less than 1:1 and no more than 1:11. The current preferred designed ratio is 1:2.2

Preferably the width ratios can be as previously stated.

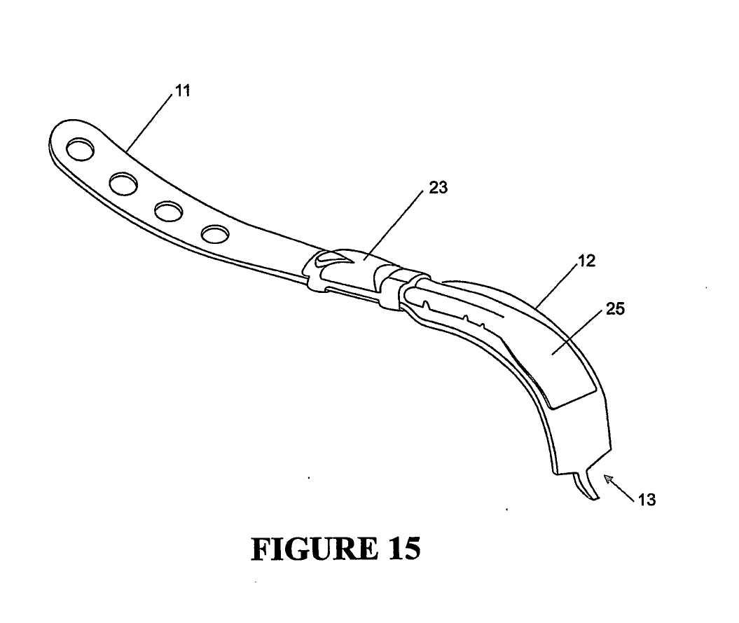

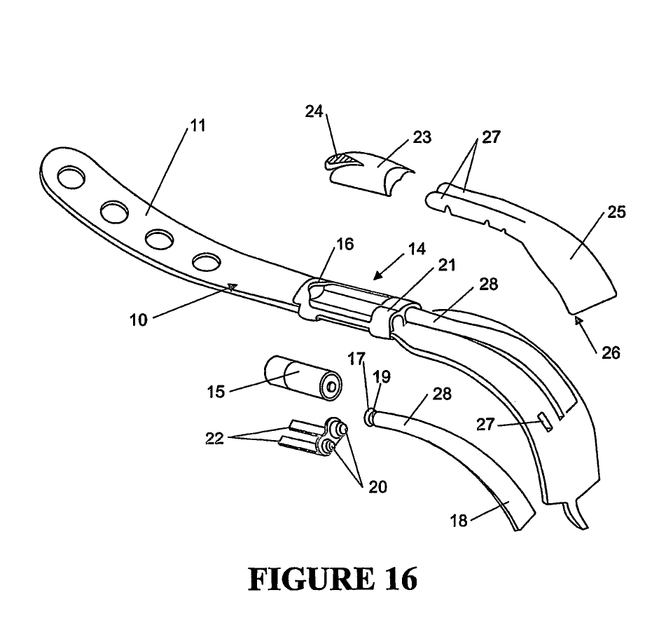

FIG. 15 and FIG. 16 show a variant of the apparatus in accordance with the present invention preferably also provided with shielding for the light duct (in this case light ducts) but which is to use a throw away battery.

In the arrangement shown there is a structural member of a retractor as previously described but with some modification to accept plural light ducts, in this case two.

In this embodiment a structural member 10 with its handle region 11, its curved region 12 and its distal region 13 is provided with a dock 14 for a throw away battery 15. The dock 14 preferably comprises a variant of the attachment apparatus previously described. This variant 16, by clamps or the like, preferably embraces and is retained by the shank region of the main structural member 13 beyond the curved region 12.

The member 16 is capable of locating the light inlet ends 17 of each light duct 18 by an annular recess 19. These light ducts, as previously described, receive from their inlet ends 17 light, in this case emitted from each of a pair of light emitting diodes (LEDs) 20 which are located in the dock 14 in part under the part 21 of the member 16. The LED assembly 20 has electrical contact members 22 extending under the battery such that a cover member 23 with appropriate electrical contacts (and optionally a switch) can co-operate in order to close a circuit through the battery 15 when it is docked with its male end contacting a contact to the LEDs 20.

Indeed a switch such as, for example, member 24 can instead be a fixed member which simply acts as a lock in component for the cover since the battery 15 is preferably thrown away after an operation and replaced after sterilisation of the remainder of the apparatus.

Shown for the two light ducts 18 is a contoured shielding member 25 having appropriate means for association with the structural member 10 and/or the component 16. As shown there are parts 26 each to associate with an opening 27 and end regions 27 capable of clamping about the regions 28 of each light duct 18 even if they do not insert into the openings 29 under the region 21 of the member 16.

Persons skilled in the art will appreciate that the present invention offers a variety of different embodiments, some to receive light from a light cable and others to generate their own light reliant on a power source that can be electrical ducted to the retractor assembly or, as described with reference to FIGS. 15 and 16, can be battery powered by a battery carried by the retractor.

1-47. (canceled)

48. A retractor useful in surgery, said retractor having
a main structural member defining
a handle region,
a distal region, and
an intermediate region, said intermediate region curving on planes normal to its main transverse dimension thereby to define a low profile form having a “concave” lower side and a “convex” upper side,
a light duct capable of ducting light it receives, said duct, at least in part away from its handle proximate inlet end, substantially conforming to the intermediate region so as to maintain a low profile thereover whilst having an emission end capable of emitting light the duct has received towards a zone in which said distal region of the main structural member is being operated,
attachment apparatus integral with or attaching to the light duct at or adjacent the inlet end of the light duct engaged with or for engagement with the main structural member, and
a shielding member attachable to at least one of
i) the attachment member,
ii) the light duct, and
iii) the intermediate member
so as, also in a low profile at least in part away from its handle proximate end, of substantially conforming to the light duct and/or the adjacent intermediate region.

49. The retractor assembly according to claim 48 in which the light duct is a moulded transparent plastics member preferably having the attachment integrally moulded therewith.

50. The retractor assembly according to claim 48 in which the light duct is a fabrication from two moulded components.

51. The retractor assembly according to claim 48 in which said light duct is adapted at its inlet end to receive light ducted via light cable, a fibre optic bundle, tube, light cable or the like.

52. The retractor assembly according to claim 48 in which the ratio between the light inlet surface area and light outlet surface area is a ratio of no less than 1:1 and no more than 1:11.

53. The retractor assembly according to claim 52 in which the ratio between the light inlet surface area and light outlet surface area is 1:2.2

54. The retractor assembly according to claim 48 in which the emission end of the light duct is substantially of a flattened section so as to better conform to the low profile of the intermediate region.

55. The retractor assembly according to claim 54 in which the flattening and broadening is such that the outlet is thinner than the inlet diameter or notional diameter and is at least as wide as two such diameters.

56. The retractor assembly according to claim 55 in which the flattening and broadening is such that the outlet is thinner than the inlet diameter or notional diameter and is at least as wide as three or more such diameters.

57. The retractor assembly according to claim 54 in which the light duct splays to said flattened form from a non flattened form at the inlet end.

58. The retractor assembly according to claim 57 in which the shielding member has a form adapted to conform closely to the flattened form of the light duct.

59. A retractor assembly suitable for hip or other joint surgery, the assembly comprising or including
a structural member capable of being used as a retractor, the structural member having, as a proximal region, a handle or manipulation control region extending through a curved region to a distal region adapted for use in a suitable patient,
at least one light ducting means carried directly or indirectly by the structural member with a light inlet in use to receive light and, reliant on internal reflection of the light, to transmit such received light to an emission zone which will cast light to a zone at and/or about said distal region, and
at least one LED or other light source carried directly or indirectly by the structural member and positioned to provide light inputs into the inlet(s) of the light ducting means, and
provision for mounting a battery and completing the operating circuit of the LED(s) or other light source(s),
wherein when assembled the light ducting means can illuminate the and/or about the distal region.

60. The retractor assembly according to claim 59 in which a shield overlays at least part of the light ducting means.

61. The retractor assembly according to claim 59 in which the completion of the operating circuit of the LED or other light source is performed by a switch.

62. The retractor assembly according to claim 60 in which the completion of the operating circuit of the LED or other light source is performed by a switch.
